(19) United States
(12) Patent Application Publication
(10) Pub. No.: US 2025/0195731 A1
(43) Pub. Date: Jun. 19, 2025

(54) METHOD FOR DETERMINING A SODIUM AND BICARBONATE ION CONCENTRATION OF A DIALYSIS FLUID AND USE OF A METHOD FOR PROPORTIONING THE DIALYSIS FLUID

(71) Applicant: B. Braun Avitum AG, Melsungen (DE)

(72) Inventor: Nicolas David Mueglich, Fuldatal (DE)

(21) Appl. No.: 18/978,092

(22) Filed: Dec. 12, 2024

(30) Foreign Application Priority Data
Dec. 13, 2023 (DE) 10 2023 134 920.1

(51) Int. Cl. A61M 1/16; G16H 20/40

(52) U.S. Cl. CPC A61M 1/1607; A61M 1/1656; G16H 20/40; A61M 2205/3317; A61M 2205/50

(57) ABSTRACT

A method is used for determining a sodium and/or bicarbonate ion concentration of a dialysis fluid, and for proportioning of the dialysis fluid. An extracorporcal blood treatment device can be used to perform the method.

### CROSS-REFERENCE TO RELATED APPLICATION

This application claims priority under 35 U.S.C. § 119 to German Application No. 10 2023 134 920.1, filed on Dec. 13, 2023, the content of which is incorporated by reference herein in its entirety.

### FIELD

The invention relates to a method for determining a sodium and bicarbonate ion concentration of a dialysis fluid, to the use of a method for proportioning of the dialysis fluid, and to the use of an extracorporeal blood treatment device for performance of a method for determining a sodium and bicarbonate ion concentration of a dialysis fluid.

### BACKGROUND

Extracorporeal blood treatment, for example haemodialysis, involves passing the blood of a patient through a dialyser, through which a dialysis fluid is passed at the same time. In the dialyser, blood and dialysis fluid are brought into contact across a semipermeable membrane, such that mass transfer between patient blood and dialysis fluid can occur. Such a dialysis treatment has the aim of detoxifying the blood and removing excess water from the body in a patient suffering from kidney failure.

The dialysis fluid consists of highly pure water, a base fluid, also referred to as base concentrate or bicarbonate concentrate, and an acid fluid, also referred to as acid concentrate. The base fluid or base concentrate usually contains sodium hydrogencarbonate (NaHCO₃). The acid fluid or acid concentrate preferably contains sodium chloride (NaCl), potassium chloride (KCl), magnesium chloride (MgCl₂), calcium chloride (CaCl₂)), glucose and an acid such as acetic acid (CH₃COOH).

Generally, metering pumps and conductivity probes are used for preparation or proportioning of the dialysis fluid. A probe measures the conductivity after the base fluid has been added to highly pure water, which can also be referred to as osmosis water, by means of a first metering pump. A further probe measures the conductivity of the entire dialysis fluid after the acid fluid has also been added by means of a further metering pump. The amounts of base fluid and acid fluid added are regulated on the basis of the conductivities measured (so-called conductivity-controlled proportioning).

The correct composition of the dialysis fluid is of great importance for the well-being and life expectancy of a patient. This is especially true with regard to sodium and bicarbonate ion concentration. The aforementioned concentrations are generally measured indirectly via conductivity in the devices for extracorporeal blood treatment. The measurement is based on a linear model which approximates the relationship between conductivity and concentration. This requires conversion factors specific for the base fluid and acid fluid (concentrate-specific conversion factors). Said factors must be determined experimentally for the particular base fluid used and particular acid fluid used by laboratory determination of the composition of the particular base fluid and particular acid fluid for a defined conductivity. Use of the measured conductivity and of the experimentally determined concentrations allows calculation of the conversion factors. However, this method has various disadvantages. For instance, the relevant laboratory tests for each base fluid and acid fluid must be performed locally in a dialysis centre. This causes significant costs. Furthermore, the quality of the conversion factors to be determined depends on the quality of the particular laboratory test. Measurement errors in determining concentrations lead to a systematic error in the composition of the dialysis fluid. A further problem is the instability of the samples required for determining concentration. As a result of carbon dioxide (CO₂) escaping into the air, the bicarbonate ion concentration becomes distorted over time. In addition, insoluble calcium carbonate (CaCO₃) precipitates, which likewise contributes to distorting the bicarbonate ion concentration. Lastly, the calculation of the conversion factors is a complex and thus time-consuming matter, which moreover requires an increased amount of training on the part of the staff involved.

EP 2 494 998 B1 discloses a dialysate concentration monitoring method in which the conductivity is shifted proportionally on the basis of a mixing ratio. The method works only in a relative manner. Therefore, if there is an initial error or an initial inaccuracy in the composition, said error is passed on with each change in the mixing ratio.

### SUMMARY

It is an object of the present invention to provide a method for determining a sodium and bicarbonate ion concentration of a dialysis fluid that at least partly avoids disadvantages known from the prior art. It is a further object of the invention to provide for the use of a method for preparation or proportioning of a dialysis fluid and the use of an extracorporeal blood treatment device for performance of a method for determining a sodium and bicarbonate ion concentration of a dialysis fluid.

According to a first aspect, the invention provides a method for determining a sodium ion concentration and bicarbonate ion concentration of a dialysis fluid, in particular for haemodialysis and/or peritoneal dialysis.

The method comprises, in particular in chronological order or not in chronological order, the following steps:
a) providing a dilute base fluid by mixing of a base fluid having a bicarbonate ion concentration (hydrogencarbonate ion concentration), in particular one that is defined, i.e. specified or known, or undefined, with water, i.e. highly pure water or osmosis water, according to a defined, i.e. specified or known, mixing ratio and providing a dilute acid fluid by mixing of an acid fluid having a sodium ion concentration, in particular one that is defined, i.e. specified or known, or undefined, and an acid concentration, in particular one that is defined, i.e. specified or known, or undefined, with water, i.e. highly pure water or osmosis water, according to a defined, i.e. specified or known, mixing ratio,
b) preparing a pre-dialysis fluid by addition, in particular continuous or discontinuous addition, of the dilute base fluid to water, i.e. highly pure water or osmosis water,
c) determining a target value for a bicarbonate conductivity of the dialysis fluid and/or pre-dialysis fluid on the basis of a bicarbonate ion concentration, in particular one that is defined, i.e. specified or known, or undefined, for the dialysis fluid and/or pre-dialysis fluid and a parameter for converting a bicarbonate ion concentration into a bicarbonate conductivity or vice versa, i.e. for converting a bicarbonate conductivity into a bicarbonate ion concentration, and determining a target value for a conductivity, in particular end conductivity or total conductivity, of the dialysis fluid on the basis of a sodium ion concentration, in particular one that is defined, i.e. specified or known, or undefined, for the dialysis fluid and a parameter for converting a sodium ion concentration into a conductivity, in particular end conductivity or total conductivity, of the dialysis fluid or vice versa, i.e. for converting a conductivity, in particular end conductivity or total conductivity, of the dialysis fluid into a sodium ion concentration,
d) measuring a conductivity of the pre-dialysis fluid,
e) comparing the conductivity of the pre-dialysis fluid measured according to step d) with the target value for the bicarbonate conductivity of the dialysis fluid and/or pre-dialysis fluid determined according to step c) and, if necessary, carrying out further addition, in particular continuous or discontinuous addition, of the dilute base fluid to the pre-dialysis fluid, in particular by means of a feed pump, until the target value for the bicarbonate conductivity of the dialysis fluid and/or pre-dialysis fluid has been reached or substantially reached (so-called closed-loop controller),
f) preparing the dialysis fluid by addition, in particular continuous or discontinuous addition, of the dilute acid fluid to the pre-dialysis fluid,
g) measuring a conductivity, in particular end conductivity or total conductivity, of the dialysis fluid,
h) comparing the conductivity of the dialysis fluid measured according to step g) with the target value for the conductivity of the dialysis fluid determined according to step c) and, if necessary, carrying out further addition, in particular continuous or discontinuous addition, of the dilute acid fluid to the dialysis fluid, in particular by means of a further feed pump, until the target value for the conductivity of the dialysis fluid has been reached or substantially reached (so-called closed-loop controller), and
i) determining the bicarbonate ion concentration of the dialysis fluid on the basis of the conductivity of the pre-dialysis fluid measured according to step d) and the parameter for converting a bicarbonate ion concentration into a bicarbonate conductivity or vice versa, i.e. for converting a bicarbonate conductivity into a bicarbonate ion concentration, and determining the sodium ion concentration of the dialysis fluid on the basis of the conductivity of the dialysis fluid measured according to step g) and the parameter for converting a sodium ion concentration into a conductivity of the dialysis fluid or vice versa, i.e. for converting a conductivity of the dialysis fluid into a sodium ion concentration,
wherein the parameter for converting a bicarbonate ion concentration into a bicarbonate conductivity or vice versa, i.e. for converting a bicarbonate conductivity into a bicarbonate ion concentration, and the parameter for converting a sodium ion concentration into a conductivity of the dialysis fluid or vice versa, i.e. for converting a conductivity of the dialysis fluid into a sodium ion concentration, are both ascertained automatically.

In the context of the present invention, the parameter for converting a bicarbonate ion concentration into a bicarbonate conductivity or vice versa can also be referred to as a conversion factor for converting a bicarbonate ion concentration into a bicarbonate conductivity or vice versa.

In the context of the present invention, the parameter for converting a sodium ion concentration into a conductivity of the dialysis fluid or vice versa can also be referred to as a conversion factor for converting a sodium ion concentration into a conductivity of the dialysis fluid or vice versa.

The expression “dialysis fluid” in the context of the present invention is to be understood to mean a fluid, in particular a ready-to-use or non-ready-to-use fluid, for dialysis, in particular haemodialysis and/or peritoneal dialysis, that consists of water, i.e. highly pure water (also referred to as osmosis water), the base fluid (also referred to as base concentrate) and the acid fluid (also referred to as acid concentrate).

The expression “base fluid” in the context of the present invention is to be understood to mean an undiluted base fluid, i.e. an undiluted bicarbonate concentrate or bicarbonate ion concentrate, having a defined bicarbonate ion concentration.

The expression “dilute base fluid” in the context of the present invention is to be understood to mean a base fluid which has been diluted or is in a diluted state, in particular with regard to the concentrations of said fluid, as a result of mixing with water, i.e. highly pure water or osmosis water, according to a defined, i.e. specified or known, mixing ratio.

The expression “acid fluid” in the context of the present invention is to be understood to mean an undiluted acid fluid, i.e. an undiluted acid-containing concentrate or acid concentrate, having a defined sodium ion concentration, in particular a defined sodium ion concentration, potassium ion concentration, magnesium ion concentration and calcium ion concentration, and a defined acid concentration, in particular acetic acid concentration.

The expression “dilute acid fluid” in the context of the present invention is to be understood to mean an acid fluid which has been diluted or is in a diluted state, in particular with regard to the concentrations of said fluid, as a result of mixing with water, i.e. highly pure water or osmosis water, according to a defined, i.e. specified or known, mixing ratio.

The expression “conductivity” in the context of the present invention means an electrical conductivity.

The expression “bicarbonate conductivity” in the context of the present invention is to be understood to mean a conductivity stemming from bicarbonate ions and sodium ions, in particular in the ratio of 1:1.

The expression “end conductivity or total conductivity of the dialysis fluid” in the context of the present invention is to be understood to mean a conductivity stemming from all ion species, in particular cation species, present in the dialysis fluid, preferably a conductivity stemming from, in particular, the sodium ions and preferably potassium ions, magnesium ions and calcium ions.

The conductivities measured according to steps d) and g) may be in particular a temperature-compensated conductivity in each case.

The expression “temperature-compensated conductivity” in the context of the present invention is to be understood to mean a conductivity corresponding to the conductivity at a room temperature or ambient temperature of 25° C.

In the context of the present invention, the expression “substantially achieved” used in the context of step e) means that the conductivity of the pre-dialysis fluid measured according to step d) may deviate ≤1%, in particular <1%, from the target value for the bicarbonate conductivity of the dialysis fluid and/or pre-dialysis fluid.

In the context of the present invention, the expression “substantially achieved” used in the context of step h) means that the conductivity of the dialysis fluid measured according to step g) may deviate ≤1%, in particular <1%, from the target value for the conductivity of the dialysis fluid.

Step c) may be performed in particular before or after step a) or step b), in particular between steps a) and b) or, preferably, between steps b) and d).

The expression “highly pure water” or osmosis water in the context of the present invention is to be understood to mean sterile water without electrolytes or without any appreciable electrolytes, i.e. with an electrolyte content of at most 0.01 mEq/l. Furthermore, the highly pure water or osmosis water may have in particular a conductivity ≤1.1 μS/cm at 20° C. and/or a concentration of bacterial endotoxins <0.25 IU/ml (approx. 25 ng/l) and/or a concentration of total organic carbon (TOC)≤0.5 mg/l and/or a nitrate concentration ≤0.2 mg/l.

The automatic ascertainment of parameters for converting conductivities into sodium and bicarbonate ion concentrations or vice versa makes it possible to minimize or avoid the disadvantages mentioned at the start in connection with conventional methods. Costly, time-consuming and error-prone laboratory tests and subsequent calculations can therefore be dispensed with. This leads to a significant simplification of the preparation for and performance of extracorporeal blood treatment and, in particular, to an increase in safety for patients to be treated.

In an embodiment of the invention, the base fluid also has a sodium ion concentration, in particular one that is defined, i.e. specified or known, or undefined.

In particular, the base fluid is provided in the form of an aqueous basic solution. Preferably, the base fluid is provided in the form of an aqueous basic solution comprising sodium bicarbonate, also referred to as sodium hydrogencarbonate (NaHCO₃). Such a solution in the context of the present invention can also be referred to as an aqueous basic sodium bicarbonate solution or sodium hydrogencarbonate solution.

The base fluid may have a bicarbonate ion concentration of 600 mmol/l to 1500 mmol/l, in particular 1000 mmol/l to 1500 mmol/l. For example, the base fluid may have a bicarbonate ion concentration of 1000 mmol/l or 1300 mmol/l.

Furthermore, the base fluid may have a sodium ion concentration of 600 mmol/l to 1500 mmol/l, in particular 1000 mmol/l to 1500 mmol/l. For example, the base fluid may have a sodium ion concentration of 1000 mmol/l or 1300 mmol/l.

The acid fluid may also have at least one further cation species concentration, in particular one that is defined, i.e. specified or known, or undefined, in particular selected from the group consisting of a potassium ion concentration, in particular one that is defined, i.e. specified or known, or undefined, a magnesium ion concentration, in particular one that is defined, i.e. specified or known, or undefined, a calcium ion concentration, in particular one that is defined, i.e. specified or known, or undefined, and combinations of at least two of the aforementioned cation species concentrations.

In a further embodiment of the invention, the acid fluid also has a potassium ion concentration, in particular one that is defined, i.e. specified or known, or undefined, a magnesium ion concentration, in particular one that is defined, i.e. specified or known, or undefined, and a calcium ion concentration, in particular one that is defined, i.e. specified or known, or undefined.

The acid of the acid fluid may be in principle an inorganic acid and/or organic acid. The acid may be in particular selected from the group consisting of hydrochloric acid, acetic acid, lactic acid, acetoacetic acid, citric acid, malic acid, maleic acid, pyruvic acid, succinic acid and combinations, in particular mixtures, of at least two of the aforementioned acids.

Preferably, the acid fluid comprises acetic acid as acid.

Furthermore, the acid fluid may also comprise an osmotically active compound. The osmotically active compound may be selected from the group consisting of glycine, monosaccharides, disaccharides, polysaccharides, sugar alcohols, gelatin, amino acids and combinations, in particular mixtures, of at least two of the aforementioned osmotically active compounds. The monosaccharides may be selected from the group consisting of glucose, fructose, galactose and combinations, in particular mixtures, of at least two of the aforementioned monosaccharides. The disaccharides may be selected from the group consisting of sucrose, maltose, trehalose and combinations, in particular mixtures, of at least two of the aforementioned disaccharides. The polysaccharides may be selected from the group consisting of dextrin, starch, polyglucose, hydroxyethyl starch and combinations, in particular mixtures, of at least two of the aforementioned polysaccharides. The sugar alcohols may be selected from the group consisting of xylitol, mannitol, sorbitol and combinations, in particular mixtures, of at least two of the aforementioned sugar alcohols. The amino acids may be in principle essential amino acids and/or non-essential amino acids.

Preferably, the acid fluid comprises glucose as osmotically active compound.

In particular, the acid fluid is provided in the form of an aqueous acidic solution. Preferably, the acid fluid is provided in the form of an aqueous acidic solution comprising sodium chloride, potassium chloride, magnesium chloride, calcium chloride and an acid, in particular acetic acid or citric acid.

Particularly preferably, the acid fluid is provided in the form of an aqueous acidic solution comprising sodium chloride, potassium chloride, magnesium chloride, calcium chloride, an osmotically active compound, in particular glucose, and an acid, in particular acetic acid or citric acid. With regard to further possible osmotically active compounds and further possible acids, full reference is made to the foregoing.

The acid fluid may have a sodium ion concentration of 95 mmol/l to 110 mmol/l, in particular 100 mmol/l to 103 mmol/l.

Furthermore, the acid fluid may have a potassium ion concentration of >0 mmol/l to 5 mmol/l, in particular 1 mmol/l to 4 mmol/l.

Furthermore, the acid fluid may have a magnesium ion concentration of >0 mmol/l to 1.5 mmol/l, in particular 0.5 mmol/l.

Furthermore, the acid fluid may have a calcium ion concentration of >0 mmol/l to 3 mmol/l, in particular 1 mmol/l to 1.5 mmol/l.

Steps d) and g) are preferably performed using conductivity probes or conductivity electrodes or other suitable conductivity measurement devices or equipment.

When performing step a), the base fluid may be mixed with the water according to a defined mixing ratio of base fluid to water of 1:30 or 1:36 and/or the acid fluid may be mixed with the water according to a defined mixing ratio of acid fluid to water of 1:34 or 1:44. Appropriate mixing ratios may advantageously be found on tags or labels of containers containing the base fluid or acid fluid. It is also advantageous that all acid types usable for extracorporeal blood treatment can be taken into consideration.

In a further embodiment of the invention, the target value for the bicarbonate conductivity of the dialysis fluid and/or pre-dialysis fluid is determined with the aid of a linear model, in particular according to the following equation (1):

$$BICLF = {c}_{bic} ( mmol / l ) \times {Λ}_{m , bic} \qquad ( 1 )$$

where
BICLF is the target value for the bicarbonate conductivity of the dialysis fluid and/or pre-dialysis fluid,
c_{bic} is the bicarbonate ion concentration for the dialysis fluid and/or pre-dialysis fluid and

Λ_{m,bic} is the parameter for converting a bicarbonate ion concentration into a bicarbonate conductivity or vice versa.

In a further embodiment of the invention, the target value for the conductivity of the dialysis fluid is determined with the aid of a linear model, in particular according to the following equation (2):

$$ENDLF = \{ [ {c}_{Na + , total} ( mmol / l ) - {c}_{bic} ( mmol / l ) ] \times {Λ}_{m , acid} \} + [ {c}_{bic} ( mmol / l ) \times {Λ}_{m , bic} ] \qquad ( 2 )$$

where
ENDLF is the target value for the conductivity of the dialysis fluid,
c_{Na+,totat} is the sodium ion concentration for the dialysis fluid,
c_{bic} is the bicarbonate ion concentration for the dialysis fluid and/or pre-dialysis fluid,
Λ_{m,acid} is the parameter for converting a sodium ion concentration into a conductivity of the dialysis fluid or vice versa
and

Λ_{m,bic} is the parameter for converting a bicarbonate ion concentration into a bicarbonate conductivity or vice versa.

In a further embodiment of the invention, the parameter for converting a bicarbonate ion concentration into a bicarbonate conductivity or vice versa and the parameter for converting a sodium ion concentration into a conductivity of the dialysis fluid or vice versa are ascertained automatically on the basis of concentrations of the dilute base fluid and/or the dilute acid fluid, preferably and in particular exclusively the dilute acid fluid. This has the advantage that the method is not subject to any restrictions with respect to the base fluid and/or acid fluid, preferably the acid fluid. The method is therefore in principle performable with all commercially available base fluids and acid fluids.

In a further embodiment of the invention, the parameter for converting a bicarbonate ion concentration into a bicarbonate conductivity or vice versa is ascertained automatically on the basis of a bicarbonate ion concentration of the dilute base fluid and an acid concentration of the dilute acid fluid.

In a further embodiment of the invention, the parameter for converting a sodium ion concentration into a conductivity of the dialysis fluid or vice versa is ascertained automatically on the basis of a bicarbonate ion concentration of the dilute base fluid, a sodium ion concentration of the dilute base fluid, a sodium ion concentration of the dilute acid fluid, a potassium ion concentration of the dilute acid fluid, a magnesium ion concentration of the dilute acid fluid, a calcium ion concentration of the dilute acid fluid and an acid concentration of the dilute acid fluid.

In a further embodiment of the invention, the parameter for converting a bicarbonate ion concentration into a bicarbonate conductivity or vice versa is determined according to the following equation (3):

$${Λ}_{m , bic} = 0.0852 \frac{mS l}{mmol cm} \frac{{c}_{b i c , 0} + {c}_{a c id , 0} [ \frac{m E q}{l} ]}{{c}_{b ic , 0}} \qquad ( 3 )$$

where

Λ_{m,bic} is the parameter for converting a bicarbonate ion concentration into a bicarbonate conductivity or vice versa,
c_{bic,0} is a bicarbonate ion concentration of the dilute base fluid and
c_{acid,0} is an acid concentration of the dilute acid fluid.

The above embodiment of the invention is based on the following assumptions/considerations:

If the dilute base fluid is provided in the form of an aqueous basic sodium bicarbonate solution, the dilute base fluid comprises, in particular substantially comprises, two types of charged constituents, namely sodium ions and bicarbonate ions. These constituents are singly charged and therefore contribute to the conductivity of the pre-dialysis fluid. If the relationship between conductivity and concentration is approximated with a linear model, this means that each charged constituent of the dilute base fluid in the pre-dialysis fluid contributes the same share to conductivity. A parameter for converting conductivity into bicarbonate ion concentration describes the contribution to conductivity per sodium bicarbonate in a defined volume element (mS/cm)/(mmol/l).

For the bicarbonate ion concentrations of, in particular approx., 35 mmol/l that are customary in extracorporeal blood treatment, in particular dialysis, the parameter for converting conductivity into bicarbonate ion concentration can be determined experimentally as 0.083 (mS/cm)/(mmol/l). However, it must be taken into account that the bicarbonate ion concentration of the pre-dialysis fluid does not correspond to the bicarbonate ion concentration of the dialysis fluid. There are two main reasons for this:

Firstly, the pre-dialysis fluid is diluted by the addition of the dilute acid fluid. The exact degree of dilution depends in particular on whether a 1+34 or a 1+44 acid fluid is used. The expression “1+34 acid fluid” in the context of the present invention means that 1 part of acid fluid and 34 parts of water, i.e. highly pure water or osmosis water, are mixed to obtain a correspondingly dilute acid fluid. Accordingly, the expression “1+44 acid fluid” in the context of the present invention means that 1 part of acid fluid and 44 parts of water, i.e. highly pure water or osmosis water, are mixed to obtain a correspondingly dilute acid fluid. Furthermore, the proportion of dilute acid fluid also depends on the sodium ion concentration set on an extracorporeal blood treatment device. The device will generally add more dilute acid fluid at higher set values for sodium ions. In total, the bicarbonate ion concentration is reduced by between 1.7% and 3.3%. On average, the bicarbonate ion concentration is reduced by, in particular approx., 2.6%. Therefore, compensation is achieved by increasing the parameter for converting conductivity into bicarbonate ion concentration by 2.6% to 0.0852 (mS/cm)/(mmol/l).

Secondly, the dilute acid fluid comprises an acid which neutralizes some of the bicarbonate ions. The products formed are carbonic acid and a salt of the acid (e.g. acetate or citrate). The conversion factor is therefore compensated a second time in order to account for this neutralization. A ready-to-use dialysis fluid usually contains a bicarbonate ion concentration of 32 mmol/l and 3 mmol/l salt of the acid, in particular acetate. Therefore, 35 mmol/l bicarbonate ions must be provided, since 3 mmol/l are neutralized by the acid, in particular acetic acid. The conversion factor is compensated to 0.0852×35/32=0.09319 (mS/cm)/(mmol/l).

In general, this yields the above equation (3).

In a further embodiment of the invention, the parameter for converting a sodium ion concentration into a conductivity of the dialysis fluid or vice versa is determined according to the following equation (4):

$${Λ}_{m , acid , k o r} = \frac{11 \frac{mS}{cm}}{{c}_{N a , a c i {d}^{+}} ( 11 \frac{mS}{cm} ) + {c}_{a c i d} ( 11 \frac{mS}{cm} )} \qquad ( 4 )$$

where
Λ_{m,acid,kor} is the parameter for converting a sodium ion concentration into a conductivity of the dialysis fluid or vice versa,

$${c}_{N a , a c i {d}^{+}} ( 11 \frac{mS}{cm} )$$

is a sodium ion concentration of the dilute acid fluid that brings about a conductivity of the dilute acid fluid of 11 mS/cm,
and

$${c}_{a c i d} ( 11 \frac{mS}{cm} )$$

is an acid concentration of the dilute acid fluid having a sodium ion concentration which brings about a conductivity of the dilute acid fluid of 11 mS/cm.

In a further embodiment of the invention, the sodium ion concentration of the dilute acid fluid that brings about a conductivity of the dilute acid fluid of 11 mS/cm is determined according to the following equation (5):

$${c}_{N a , a c i {d}^{+}} ( 11 \frac{mS}{cm} ) = \frac{11 \frac{mS}{cm}}{{Λ}_{m , acid}} \qquad ( 5 )$$

where

$${c}_{N a , a c i {d}^{+}} ( 11 \frac{mS}{cm} )$$

is the sodium ion concentration of the dilute acid fluid that brings about a conductivity of the dilute acid fluid of 11 mS/cm, and

Λ_{m,acid} is an uncorrected parameter for converting a sodium ion concentration into a conductivity, in particular end conductivity or total conductivity, of the dialysis fluid or vice versa, i.e. for converting a conductivity, in particular end conductivity or total conductivity, of the dialysis fluid into a sodium ion concentration.

In a further embodiment of the invention, the uncorrected parameter Λ_{m,acid} for converting a sodium ion concentration into a conductivity of the dialysis fluid or vice versa is determined according to the following equation (6):

$${Λ}_{m , acid} = 0.0 \; 9921 \frac{mS l}{m mol cm} \frac{{c}_{{Na}^{+} , 0} + {c}_{{K}^{+} , 0} + 2 * {c}_{{Mg}^{+} , 0} + 2 * {c}_{{Ca}^{+} , 0}}{{c}_{{Na}^{+} , 0}} \qquad ( 6 )$$

where

Λ_{m,acid} is the uncorrected parameter for converting a sodium ion concentration into a conductivity of the dialysis fluid or vice versa,
c_{Nat}_{⁺}_{,0} is a sodium ion concentration of the dilute acid fluid,
c_{K}_{⁺}_{,0} is a potassium ion concentration of the dilute acid fluid,
2*c_{Mg}_{⁺}_{,0} is a magnesium ion concentration of the dilute acid fluid and
2*c_{Ca}_{⁺}_{,0} is a calcium ion concentration of the dilute acid fluid.

In a further embodiment of the invention, the acid concentration of the dilute acid fluid having a sodium ion concentration which brings about a conductivity of the dilute acid fluid of 11 mS/cm is determined according to the following equation (7):

$${c}_{a c i d} ( 11 \frac{mS}{cm} ) = \frac{{c}_{a c id , 0} [ \frac{m E q}{l} ] * {c}_{N a , a c i {d}^{+}} ( 11 \frac{mS}{cm} )}{{c}_{N {a}^{+} , 0} - {c}_{b i c , 0} - {c}_{a c id , 0} [ \frac{m E q}{l} ]} \qquad ( 7 )$$

where

$${c}_{a c i d} ( 11 \frac{mS}{cm} )$$

is an acid concentration of the dilute acid fluid having a sodium ion concentration which leads to a conductivity of 11 mS/cm,
c_{bic,0} is a bicarbonate ion concentration of the dilute base fluid,
c_{acid,0} is an acid concentration of the dilute acid fluid,
c_{Na}_{⁺}_{,0} is a sodium ion concentration of the dilute acid fluid and

$${c}_{N a , a c i {d}^{+}} ( 11 \frac{mS}{cm} )$$

is a sodium ion concentration of the dilute acid fluid that brings about a conductivity of the dilute acid fluid of 11 mS/cm.

The four preceding embodiments of the invention are based on the following assumptions/considerations:

The dilute acid fluid preferably comprises sodium, potassium, magnesium and calcium ions, in particular as constituent of sodium chloride, potassium chloride, magnesium chloride and calcium chloride. Thus, the aforementioned components contribute to the conductivity of the dilute acid fluid.

In principle, the acid concentration of the dilute acid fluid hardly contributes to conductivity, since the acid of the dilute acid fluid is generally an acid which only undergoes slight dissociation in aqueous solution. However, the dilute acid fluid is added to the pre-dialysis fluid comprising bicarbonate ions for preparation of the dialysis fluid, thereby resulting in an acid-base reaction. The resultant salt of the acid, for example an acetate salt, contributes to conductivity. In contrast to this, the neutralization of the bicarbonate ions (by reaction with the acid) leads to the formation of carbonic acid which makes little or no contribution to conductivity. As a result, there is therefore little or no change in net conductivity, and so this contribution can be disregarded.

Since the bicarbonate has already been added when the dilute acid fluid is added to the pre-dialysis fluid, two factors are relevant to total conductivity: the contribution by the bicarbonate ions, which was previously determined, and the contribution by the dilute acid fluid. The contribution by the dilute acid fluid is likewise approximated via a linear model, in which it is assumed that each component of the dilute acid fluid contributing to conductivity makes the same contribution to conductivity. Normalization is carried out to the desired sodium ion concentration.

In principle, the contribution to conductivity by the dilute acid fluid can be determined according to the following equation:

Contribution to conductivity by the dilute acid fluid=Sodium ion concentration of the dilute acid fluid×Parameter for converting a sodium ion concentration into a conductivity of the dialysis fluid or vice versa.

As described above, it is not only the sodium chloride in the dilute acid fluid, but optionally also other electrolytes, preferably potassium chloride, magnesium chloride and calcium chloride, that contribute to conductivity. The contribution to conductivity by the dilute acid fluid per desired sodium ion is thus higher, the greater the proportion of the other electrolytes. Therefore, in principle, the parameter for converting a sodium ion concentration into a conductivity of the dialysis fluid or vice versa varies depending on the proportions of the electrolytes.

In the context of the present invention, it is assumed (simplistically) that the contribution to conductivity by each electrolyte is identical. Experimentally, it was determined that the contribution to conductivity by sodium chloride at a concentration of, in particular approx., 140 mmol/l that is typical for extracorporeal blood treatment, in particular dialysis, is 0.09921 (mS/cm)/(mmol/l).

The method described here (so far) therefore defines a parameter for converting concentration into conductivity or vice versa according to the above equation (6) for the dilute acid fluid.

However, the aforementioned conversion parameter requires a further compensation factor. This is because the total sodium ion concentration is the result of two contributions, namely the sodium ion contribution from the dilute acid fluid and the sodium ion contribution from the dilute base fluid. For the sodium ion contribution from the dilute base fluid, the prior method assumes that one sodium ion is added for each bicarbonate ion. However, this is not completely correct. The above-mentioned neutralization with the acid of the dilute acid fluid reduces the proportion of bicarbonate ions in the dialysis fluid. This has been taken into account in the context of the present invention for the parameter for converting a bicarbonate ion concentration into a bicarbonate conductivity or vice versa by means of a compensation calculation. However, the proportion of sodium ions due to the dilute base fluid is not affected by the neutralization that occurs when the dilute acid fluid is added to the pre-dialysis fluid. Ultimately, the proportion of sodium ions added by the dilute base fluid is therefore greater than the proportion of bicarbonate ions. This additional proportion of sodium ions can be taken into account by appropriately modifying the parameter for converting concentration into conductivity or vice versa for the dilute acid fluid.

This is achieved by first calculating a sodium ion concentration which—taken individually—leads to a conductivity of 11 mS/cm. This conductivity value typically corresponds to the contribution by the dilute acid fluid to the conductivity of a dialysis fluid having a total conductivity of 14 mS/cm, of which a conductivity of 3 mS/cm is due to the bicarbonate ions. This sodium ion concentration may be determined according to the above equation (5):

The proportional model assumes that one sodium ion is added for one bicarbonate ion. However, this does not apply to bicarbonate ions after reaction with the acid of the dilute acid fluid. In reality, for each bicarbonate ion eliminated by the acid, an additional sodium ion is added.

Therefore, in a third step, the acid concentration of the dilute acid fluid having a sodium ion concentration which makes a contribution to conductivity of 11 mS/cm for a dialysis fluid having a total conductivity of 14 mS/cm is ascertained according to the above equation (7).

The parameter for converting a sodium ion concentration into a conductivity of the dialysis fluid or vice versa may be calculated in a fourth step according to the above equation (4), i.e. by division of 11 mS/cm by the sum total of the sodium ions added by the dilute acid fluid, corrected by the amount of sodium ions that is disregarded owing to the acid-base reaction between the bicarbonate of the dilute base fluid and the acid of the dilute acid fluid.

Preferably, when performing step i), the bicarbonate ion concentration of the dialysis fluid is determined with the aid of a linear model, in particular according to the following equation (8):

$${c}_{bic}^{+} ( m mol / l ) = {BICLF}^{+} / {Λ}_{m , bic} \qquad ( 8 )$$

where
c⁺_{bic} is the bicarbonate ion concentration of the dialysis fluid,
BICLF⁺ is the conductivity of the pre-dialysis fluid measured according to step d), and

Λ_{m,bic} is the parameter for converting a bicarbonate ion concentration into a bicarbonate conductivity or vice versa.

More preferably, when performing step i), the sodium ion concentration of the dialysis fluid is determined with the aid of a linear model, in particular according to the following equation (9):

$${c}_{N a + , total}^{+} ( mmol / l ) = \{ E N D L {F}^{+} - [ {c}_{bic}^{+} ( mmol / l ) \times {Λ}_{m , bic} ] \} / {Λ}_{m , a c i d , k o r} + {c}_{bic}^{+} ( mmol / l ) \qquad ( 9 )$$

where
c⁻_{Na+,total} is the sodium ion concentration of the dialysis fluid,
ENDLF⁺ is the conductivity of the dialysis fluid measured according to step g),
c⁺_{bic} is the bicarbonate ion concentration of the dialysis fluid,
   Λ_{m,acid,kor} is the parameter for converting a sodium ion concentration into a conductivity of the dialysis fluid or vice versa
   and
   Λ_{m,bic} is the parameter for converting a bicarbonate ion concentration into a bicarbonate conductivity or vice versa.

Alternatively, the parameter for converting a bicarbonate ion concentration into a bicarbonate conductivity or vice versa may be determined according to the following equation (3 #):

$${Λ}_{m , bic} = 0.0852 \frac{mS l}{mmol cm} \qquad ( 3 \# )$$

where
Λ_{m,bic} is the parameter for converting a bicarbonate ion concentration into a bicarbonate conductivity or vice versa.

In this case, the parameter for converting a bicarbonate ion concentration into a bicarbonate conductivity or vice versa is therefore independent of the dilute base fluids and/or dilute acid fluids used, in particular the dilute acid fluids used.

Furthermore, the parameter for converting a sodium ion concentration into a conductivity of the dialysis fluid or vice versa may be determined according to the following equation (4 #):

$${Λ}_{m , acid} = 0.0 \; 9921 \frac{mS l}{m mol cm} \frac{{c}_{{Na}^{+} , 0} + {c}_{{K}^{+} , 0} + 2 * {c}_{{Mg}^{+} , 0} + 2 * {c}_{{Ca}^{+} , 0}}{{c}_{{Na}^{+} , 0}} \qquad ( 4 \# )$$

where
Λ_{m,acid} is the parameter for converting a sodium ion concentration into a conductivity of the dialysis fluid or vice versa,
c_{Na}_{⁺}_{,0} is a sodium ion concentration of the dilute acid fluid,
c_{K}_{⁺}_{,0} is a potassium ion concentration of the dilute acid fluid,
2*c_{Mg}_{⁺}_{,0} is a magnesium ion concentration of the dilute acid fluid and
2*c_{Ca}_{⁺}_{,0} is a calcium ion concentration of the dilute acid fluid.

In this case, when performing step i), the sodium ion concentration of the dialysis fluid is determined with the aid of a linear model, in particular according to the following equation (9 #):

$${c}_{N a + , total}^{+} ( mmol / l ) = \{ E N D L {F}^{+} - [ {c}_{bic}^{+} ( mmol / l ) \times {Λ}_{m , bic} ] \} / {Λ}_{m , acid} + {c}_{bic}^{+} ( mmol / l ) \qquad ( 9 \# )$$

where
c*_{Na+,total} is the sodium ion concentration of the dialysis fluid,
ENDLF⁺ is the conductivity of the dialysis fluid measured according to step g),
c⁺_{bic} is the bicarbonate ion concentration of the dialysis fluid,
   Λ_{m,acid} is the parameter for converting a sodium ion concentration into a conductivity of the dialysis fluid or vice versa
   and
   Λ_{m,bic} is the parameter for converting a bicarbonate ion concentration into a bicarbonate conductivity or vice versa.

The set value for the bicarbonate ion concentration input by a user into an extracorporeal blood treatment device, in particular dialysis machine, is evaluated by the extracorporeal blood treatment device as bicarbonate ion concentration after neutralization with the acid of the dilute acid fluid, i.e. as bicarbonate ion concentration of the dialysis fluid, c_{BIC,post}. From this concentration, the bicarbonate ion concentration before neutralization with the acid of the dilute acid fluid, i.e. the bicarbonate ion concentration of the pre-dialysis fluid, c_{BIC,pre} is determined according to the following equation (1*):

$${c}_{bic , pre} = \frac{{c}_{bic , post} + \frac{{c}_{Na , set}}{{c}_{{Na}^{+} , 0} - {c}_{bic , 0} - {c}_{acid , 0}} {c}_{acid , 0}}{1 + \frac{{c}_{Na , set}}{{c}_{{c}_{{Na}^{+} , 0}} - {c}_{bic , 0} - {c}_{acid , 0}}} \qquad ( {1}^{*} )$$

where
c_{bic,pre} is the bicarbonate ion concentration of the pre-dialysis fluid,
c_{bic,post} is a bicarbonate ion concentration specified for the dialysis fluid,
c_{Na,set} is a sodium ion concentration specified for the dialysis fluid,
c_{bic,0} is a bicarbonate ion concentration of the dilute base fluid,
c_{Na}_{⁺}_{,0} is a sodium ion concentration of the dilute acid fluid and
c_{acid,0} is an acid concentration of the dilute acid fluid.

The value c_{BIC,pre} is then inserted into equation (3) as set value for the bicarbonate ion concentration. This results in the parameter for converting a bicarbonate ion concentration into a bicarbonate conductivity or vice versa independently of the dilute base fluid and/or dilute acid fluid used, in particular dilute acid fluid, according to the above equation (3 #). The parameter for converting a sodium ion concentration into a conductivity of the dialysis fluid or vice versa is obtained according to the above equation (4 #).

As a result, the actual concentration of further cation species of the dialysis fluid, in particular selected from the group consisting of potassium ion concentration, magnesium ion concentration, calcium ion concentration and combinations of at least two of the aforementioned cation species concentrations, at a bicarbonate and sodium ion concentration specified for the dialysis fluid may advantageously be determined according to the following equation (10):

$${c}_{X , act} = \frac{{c}_{N a , set} - {c}_{b i c , p r e}}{{c}_{N {a}^{+} , 0} - {c}_{b i c , 0} - {c}_{a c i d , 0}} {c}_{X , 0} \qquad ( 10 )$$

where
c_{X,act} is the actual concentration of a further cation species of the dialysis fluid, in particular selected from the group consisting of potassium ion concentration, magnesium ion concentration, calcium ion concentration and combinations of at least two of the aforementioned cation species concentrations,
c_{Na,set} is a sodium ion concentration specified for the dialysis fluid,
c_{bic,pre} is the bicarbonate ion concentration of the pre-dialysis fluid,
c_{X,0} is a concentration of a further cation species of the dilute acid fluid, in particular selected from the group consisting of potassium ion concentration, magnesium ion concentration, calcium ion concentration and combinations of at least two of the aforementioned cation species concentrations,
c_{Na}_{⁺}_{,0} is a sodium ion concentration of the dilute acid fluid,
c_{acid,0} is an acid concentration of the dilute acid fluid and
c_{bic,0} is a bicarbonate ion concentration of the dilute base fluid.

Furthermore, the total buffer of the dialysis fluid at a bicarbonate and sodium ion concentration specified for the dialysis fluid may advantageously be determined.

The total buffer may be specified according to the dilute acid fluid selected:

Acetic acid and citric acid (example: SW 380, Citradial):

In this case, the total buffer is identical to the bicarbonate ion concentration before neutralization with the acid of the dilute acid fluid, i.e. to the bicarbonate ion concentration of the pre-dialysis fluid, since one acid salt counterpart is created for each neutralized bicarbonate:

$$Total buffer = {c}_{BIC , pre} \qquad ( 11 )$$

Hydrochloric acid (example: Lympha)

In this case, the total buffer is identical to the bicarbonate ion concentration after neutralization with the acid of the dilute acid fluid, i.e. to the bicarbonate ion concentration of the dialysis fluid. In the case of hydrochloric acid, the salt of the acid formed is merely chloride, which is not metabolized into bicarbonate in the body like acetate and citrate.

$$Total buffer = {c}_{bic , post} \qquad ( 12 )$$

Acetoacetic acid (example: Granuflo)

In this case, for each acetic acid added, there is added a further acetate which can be metabolized to bicarbonate.

$$Total buffer = {c}_{bic , pre} + \frac{{c}_{Na , set} - {c}_{bic , pre}}{{c}_{{Na}^{+} , 0} - {c}_{bic , 0} - {c}_{acid , 0}} {c}_{acid , 0} \qquad ( 13 )$$

where
c_{bic,pre} is the bicarbonate ion concentration of the pre-dialysis fluid,
c_{Na,set} is a sodium ion concentration specified for the dialysis fluid,
c_{Na}_{⁺}_{,0} is a sodium ion concentration of the dilute acid fluid,
c_{acid,0} is an acid concentration of the dilute acid fluid and
c_{bic,0} is a bicarbonate ion concentration of the dilute base fluid.

The method may therefore comprise a further step j) determining the concentration of at least one further constituent of the dialysis fluid, wherein the at least one further constituent is selected from the group consisting of potassium ions, magnesium ions, calcium ions, total buffer and combinations of at least two of the aforementioned constituents of the dialysis fluid.

In a further embodiment of the invention, the concentrations of the base fluid and/or the acid fluid are stored or saved on an extracorporeal blood treatment device, in particular on a haemodialysis device or peritoneal dialysis device, preferably on a dialysis machine, in particular haemodialysis machine or peritoneal dialysis machine, or some other system.

In a further embodiment of the invention, at least part of the method, in particular only part of the method or the entire method, is performed by an extracorporeal blood treatment device, in particular a haemodialysis device or peritoneal dialysis device, preferably a dialysis machine, in particular haemodialysis machine or peritoneal dialysis machine.

Furthermore, it may be preferable to perform the method only semi-automatically or fully automatically. In other words, it may be especially preferable if only a number of steps, i.e. only some steps, or all the steps of the method are performed automatically.

In a further embodiment of the invention, depending on the bicarbonate ion concentration determined according to step i), a further addition, in particular continuous or discontinuous addition, of the dilute base fluid to the pre-dialysis fluid is carried out and/or, depending on the sodium ion concentration determined according to step i), a further addition, in particular continuous or discontinuous addition, of the dilute acid fluid to the dialysis fluid is carried out.

In a further embodiment of the invention, the dialysis fluid is prepared or proportioned by the aid of the method or haemodialysis is carried out by the aid of the method.

According to a second aspect, the invention provides for the use of the method according to the first aspect of the invention for preparation or proportioning of a/the dialysis fluid, in particular for haemodialysis and/or peritoneal dialysis, preferably haemodialysis.

Regarding further features and advantages of the use, in particular in relation to the method, full reference is made to the discussions with respect to the first aspect of the invention. The features and advantages that they describe in relation to the method also apply, mutatis mutandis, to the use according to the second aspect of the invention.

According to a third aspect, the invention provides for an extracorporeal blood treatment device, in particular haemodialysis device and/or peritoneal dialysis device, preferably haemodialysis device, in particular for performance of a method according to the first aspect of the invention, or for the use of an extracorporeal blood treatment device, in particular haemodialysis device and/or peritoneal dialysis device, preferably haemodialysis device, for performance of a method according to the first aspect of the invention.

The device may in particular comprise a first mixing chamber for mixing of water, i.e. highly pure water or osmosis water, and a dilute base fluid to form a pre-dialysis fluid and a second mixing chamber for mixing of the pre-dialysis fluid and a dilute acid fluid to form a dialysis fluid.

Furthermore, the device may comprise a first feed pump for feeding of the dilute base fluid into the first mixing chamber and a second feed pump for feeding of the dilute acid fluid into the second mixing chamber.

Furthermore, the device may comprise a temperature sensor for measurement of a temperature of the pre-dialysis fluid and a conductivity sensor for measurement of a conductivity of the pre-dialysis fluid.

Furthermore, the device may comprise a temperature sensor for measurement of a temperature of the dialysis fluid and a conductivity sensor for measurement of a conductivity of the dialysis fluid.

Furthermore, the device may comprise software for comparison of a measured conductivity of the pre-dialysis fluid with a target value for a bicarbonate conductivity of the dialysis fluid and/or pre-dialysis fluid and for comparison of a measured conductivity of the dialysis fluid with a target value for a conductivity of the dialysis fluid.

Furthermore, the device may comprise a controller for control of a feed rate of the dilute base fluid into the first mixing chamber according to a measured conductivity of the pre-dialysis fluid and for control of a feed rate of the dilute acid fluid into the second mixing chamber according to a measured conductivity of the dialysis fluid.

The aforementioned components of the device may in particular be part of a dialysis fluid providing unit of the device.

Preferably, the extracorporeal blood treatment device is in the form of a dialysis machine, in particular for haemodialysis and/or peritoneal dialysis, preferably haemodialysis.

Regarding further features and advantages of the device or use of the device, in particular in relation to the method, full reference is made to the discussions with respect to the first aspect of the invention. The features and advantages that they describe in relation to the method also apply, mutatis mutandis, to the device or use of the device according to the third aspect of the invention.

The advantages of the method according to the invention shall be summarized here once again as follows:

By means of the method according to the invention, conversion factors for base fluids (base concentrates or bicarbonate concentrates) and acid fluids (acid concentrates) can be determined merely on the basis of the standard concentration of the base fluids and/or acid fluids, in particular only the acid fluids, without any experimental laboratory determination. The factors are optimized to the standard concentrations of the base fluids and/or acid fluids, in particular only the acid fluids. The experimentally determined model error in this range is advantageously less than 1 mmol/l for sodium and bicarbonate ions.

Conversion factors, which previously had to be determined by a technician, therefore no longer need to be saved in an extracorporeal blood treatment device, in particular a dialysis machine. Rather, only the standard concentrations of the base fluids and/or acid fluids used, in particular the acid fluids, need to be configured in the machine. Such an activity does not require a trained technician and can be performed, for example, by a dialysis nurse.

Further features and advantages of the invention will become apparent from the claims and from the following description of preferred exemplary embodiments of the invention which are shown schematically with the aid of the drawings. It is understood that the invention is not to be limited thereto.

### BRIEF DESCRIPTION OF THE DRAWINGS

FIG. 1 shows a dialysis fluid providing unit of a dialysis machine for performance of the method according to the invention,

FIG. 2 shows a closed-loop controller for setting of a feed rate of a pump for feeding of the dilute base fluid,

FIG. 3 shows a closed-loop controller for setting of a feed rate of a pump for feeding of the dilute acid fluid,

FIG. 4 shows the determination of the target values for the bicarbonate conductivity of the pre-dialysis fluid and of the conductivity of the dialysis fluid,

FIG. 5 shows a user interface of a dialysis machine for performance of the method according to the invention, and

FIG. 6 shows a flow chart of a method according to the invention.

### DETAILED DESCRIPTION

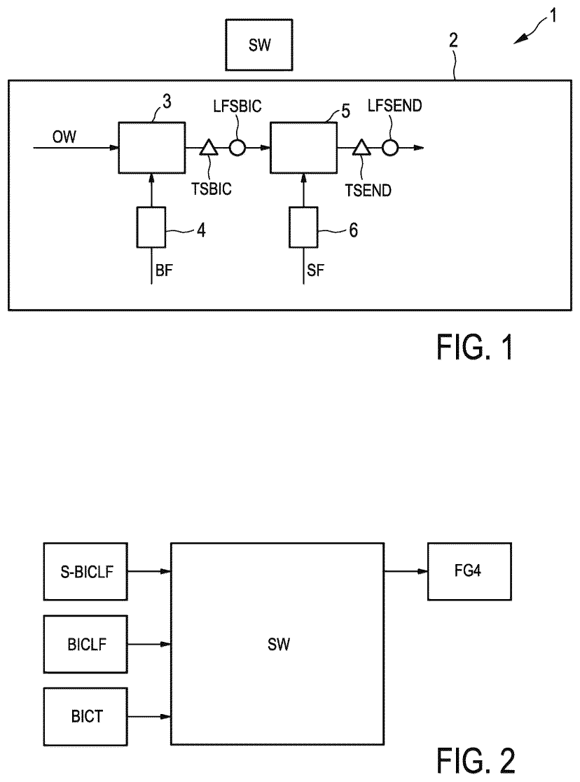

FIG. 1 shows schematically one embodiment of a device 1 for performance of the method according to the invention.

The device 1 comprises a dialysis fluid providing unit 2.

Mixed in the dialysis fluid providing unit 2 to form a dialysis fluid are osmosis water, a base fluid diluted by mixing of a base fluid having a defined or undefined bicarbonate ion concentration with water, and an acid fluid diluted by mixing of an acid fluid having a defined or undefined sodium ion concentration and a defined or undefined acid concentration with water. The dialysis fluid is, optionally at a later time, fed into a dialyser (not shown) of the device 1.

The providing or preparation of the dialysis fluid is achieved as follows:

First, the osmosis water OW and the dilute base fluid BF are mixed in a first mixing chamber 3 of the dialysis fluid providing unit 2. To this end, the dilute base fluid BF is fed into the first mixing chamber 3 via a feed pump 4, in particular one that is volumetrically controlled. In the first mixing chamber 3, ideal mixing occurs between the osmosis water OW and the dilute base fluid BF to form a pre-dialysis fluid. The pre-dialysis fluid then passes a temperature sensor TSBIC and a conductivity sensor LFSBIC. The temperature sensor TSBIC is designed to measure a temperature BICT of the pre-dialysis fluid. The conductivity sensor LFSBIC is designed to measure a conductivity BICLF of the pre-dialysis fluid. Preferably, the measurement values BICT and BICLF determined by means of the aforementioned sensors are evaluated by software SW in order to determine a temperature-compensated conductivity of the pre-dialysis fluid.

Later in the method according to the invention, the conductivity is compared with a target value for the bicarbonate conductivity S-BICLF of the dialysis fluid and/or pre-dialysis fluid by means of the software SW. Depending on the conductivity BICLF measured, the feed rate FG4 of the feed pump 4 is continuously adjusted via the software SW in order to bring the conductivity BICLF of the pre-dialysis fluid as close as possible to the target value for the bicarbonate conductivity S-BICLF (see the closed-loop controller shown schematically in FIG. 2).

In a next step, the pre-dialysis fluid is mixed with the dilute acid fluid SF in a second mixing chamber 5 of the dialysis fluid providing unit 2. To this end, the dilute acid fluid SF is fed into the second mixing chamber 5 via a feed pump 6, in particular one that is volumetrically controlled. In the second mixing chamber 5, ideal mixing occurs between the pre-dialysis fluid and the dilute acid fluid SF to form the dialysis fluid. The dialysis fluid then passes a temperature sensor TSEND and a conductivity sensor LFSEND. The temperature sensor TSEND is designed to measure a temperature ENDT of the dialysis fluid. The conductivity sensor LFSEND is designed to measure a conductivity ENDLF of the dialysis fluid. Preferably, the measurement values ENDT and ENDLF determined via the two aforementioned sensors are evaluated by the software SW in order to determine a temperature-compensated conductivity ENDLF.

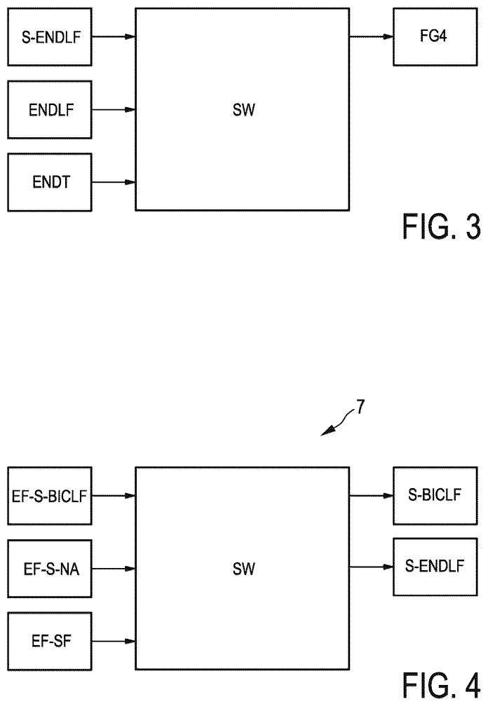

Later in the method according to the invention, the conductivity ENDLF of the dialysis fluid is compared with a target value for the conductivity S-ENDLF of the dialysis fluid by means of the software SW. Depending on the conductivity ENDLF measured, the feed rate FG6 of the feed pump 6 is continuously adjusted via the software SW in order to bring the conductivity ENDLF as close as possible to the target value for the conductivity S-ENDLF of the dialysis fluid (see the closed-loop controller shown in FIG. 3).

Preferably, the software and sensor system of the device 1 may have dual-channel redundancy for safety reasons (a second channel is not shown in FIGS. 2 and 3 for the sake of clarity).

The target values for the bicarbonate conductivity and the conductivity of the dialysis fluid that are required for the two closed-loop controllers are determined by the software SW by means of the equations (1) and (2) mentioned in the general description. The input used to this end are target concentrations for sodium ions and bicarbonate ions and the desired base fluids and acid fluids, which may be input into the device 1, specifically for performance of extracorporeal blood treatment, by a user of the device 1.

To this end, the device 1 may comprise a corresponding user interface. A suitable user interface 7 is shown schematically in FIG. 4. The user interface 7 comprises an input field EF-S-BICLF for a target value of the bicarbonate ion concentration of the dialysis fluid and/or the pre-dialysis fluid, an input field EF-S-NA for a target value of the sodium ion concentration of the dialysis fluid, and an input field EF-SF for the dilute acid fluid. From the aforementioned target values, the software SW then ascertains a target value for the bicarbonate conductivity S-BICLF of the pre-dialysis fluid and a target value for the conductivity S-ENDLF of the dialysis fluid.

Any further parameters required for calculation of the target value for the bicarbonate conductivity S-BICLF and the target value for the conductivity S-ENDLF of the dialysis fluid may advantageously be saved in the device 1, and so they do not have be input with each treatment. For example, appropriate further parameters might be available in the software SW ex factory or might be configured on-site prior to treatment.

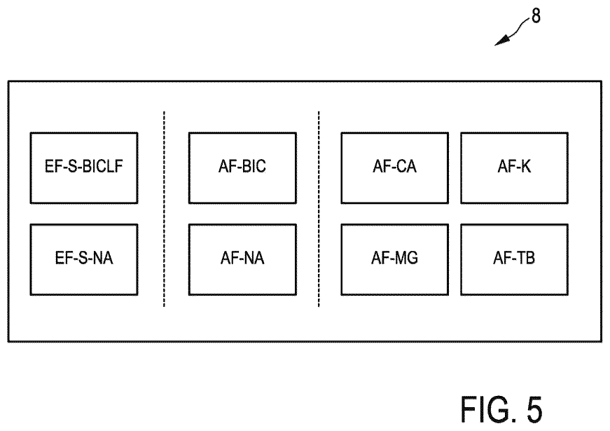

From the measured conductivities, in particular temperature-compensated conductivities, the current concentrations of the constituents of the dialysis fluid can be calculated. These in turn may be displayed on a user interface of device 1 in addition to setting parameters. An appropriate user interface is shown schematically in FIG. 5. The user interface 8 shown in FIG. 5 comprises an input field EF-S-BICLF for a target value of the bicarbonate ion concentration of the dialysis fluid and/or pre-dialysis fluid, an input field EF-S-NA for a target value of the sodium ion concentration of the dialysis fluid, a display field AF-BIC for a current bicarbonate ion concentration of the dialysis fluid and/or pre-dialysis fluid, and a display field AF-NA for a current sodium ion concentration of the dialysis fluid. Optionally, the user interface may moreover comprise a display field AF-CA for a current calcium ion concentration of the dialysis fluid, a display field AF-K for a current potassium ion concentration of the dialysis fluid, a display field AF-MG for a current magnesium ion concentration of the dialysis fluid, and a display field AF-TB for a current total buffer concentration of the dialysis fluid.

To this end, use may be made of the equations (1), (2), (10), (11), (12) and (13) stated in the general description.

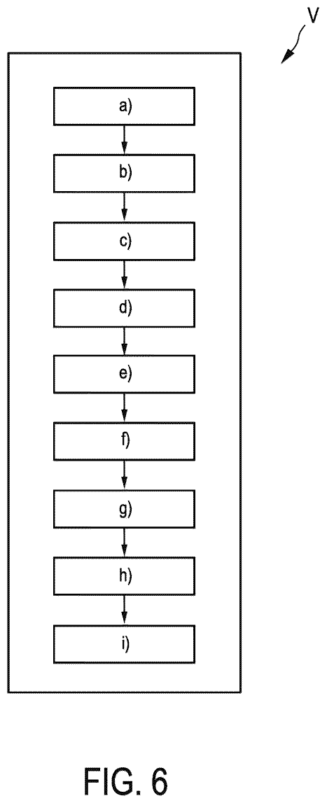

FIG. 6 shows a flow chart or flow diagram of the method V according to the invention.

The method V according to the invention is a method for determining a sodium ion concentration and bicarbonate ion concentration of a dialysis fluid, in particular for haemodialysis and/or peritoneal dialysis.

The method comprises the following steps. The following steps may be performed in chronological order or—in particular at least some of the steps—not in chronological order.

A step a) comprises providing a dilute base fluid by mixing of a base fluid having a bicarbonate ion concentration (hydrogencarbonate ion concentration), in particular one that is defined or undefined, with water, i.e. highly pure water or osmosis water, according to a defined mixing ratio and a dilute acid fluid by mixing of an acid fluid having a sodium ion concentration, in particular one that is defined or undefined, and an acid concentration, in particular one that is defined or undefined, with water, i.e. highly pure water or osmosis water, according to a defined mixing ratio.

Besides the bicarbonate ion concentration, the base fluid may also have a sodium ion concentration, in particular one that is defined or undefined. In particular, the base fluid may be provided in the form of an aqueous sodium hydrogencarbonate solution.

Besides the sodium ion concentration, the acid fluid may also have a potassium ion concentration, in particular one that is defined or undefined, a magnesium ion concentration, in particular one that is defined or undefined, and a calcium ion concentration, in particular one that is defined or undefined.

In particular, the acid fluid may be provided in the form of an aqueous acidic solution comprising sodium chloride, potassium chloride, magnesium chloride, calcium chloride and an acid, in particular acetic acid or citric acid. Optionally, the acid fluid may additionally comprise an osmotically active compound, in particular glucose.

A step b) comprises preparing a pre-dialysis fluid by addition, in particular continuous or discontinuous addition, of the dilute base fluid to water, i.e. highly pure water or osmosis water.

A step c) comprises determining a target value for a bicarbonate conductivity of the dialysis fluid and/or pre-dialysis fluid on the basis of a bicarbonate ion concentration, in particular one that is defined or undefined, for the dialysis fluid and/or pre-dialysis fluid and a parameter for converting a bicarbonate ion concentration into a bicarbonate conductivity or vice versa, i.e. for converting a bicarbonate conductivity into a bicarbonate ion concentration, and a target value for a conductivity, in particular end conductivity or total conductivity, of the dialysis fluid on the basis of a sodium ion concentration, in particular one that is defined or undefined, for the dialysis fluid and a parameter for converting a sodium ion concentration into a conductivity, in particular end conductivity or total conductivity, of the dialysis fluid or vice versa, i.e. for converting a conductivity, in particular end conductivity or total conductivity, of the dialysis fluid into a sodium ion concentration.

A step d) comprises measuring a conductivity of the pre-dialysis fluid.

A step e) comprises comparing the conductivity of the pre-dialysis fluid measured according to step d) with the target value for the bicarbonate conductivity of the dialysis fluid and/or pre-dialysis fluid determined according to step c) and, if necessary, carrying out further addition, in particular continuous or discontinuous addition, of the dilute base fluid to the pre-dialysis fluid, in particular by means of a feed pump, until the target value for the bicarbonate conductivity of the dialysis fluid and/or pre-dialysis fluid has been reached or substantially reached.

A step f) comprises preparing the dialysis fluid by addition, in particular continuous or discontinuous addition, of the dilute acid fluid to the pre-dialysis fluid.

A step g) comprises measuring a conductivity, in particular end conductivity or total conductivity, of the dialysis fluid.

A step h) comprises comparing the conductivity of the dialysis fluid measured according to step g) with the target value for the conductivity of the dialysis fluid determined according to step c) and, if necessary, carrying out further addition, in particular continuous or discontinuous addition, of the dilute acid fluid to the dialysis fluid, in particular by means of a further feed pump, until the target value for the conductivity of the dialysis fluid has been reached or substantially reached.

A step i) comprises determining the bicarbonate ion concentration of the dialysis fluid on the basis of the conductivity of the pre-dialysis fluid measured according to step d) and the parameter for converting a bicarbonate ion concentration into a bicarbonate conductivity or vice versa, i.e. for converting a bicarbonate conductivity into a bicarbonate ion concentration, and the sodium ion concentration of the dialysis fluid on the basis of the conductivity of the dialysis fluid measured according to step g) and the parameter for converting a sodium ion concentration into a conductivity of the dialysis fluid or vice versa, i.e. for converting a conductivity of the dialysis fluid into a sodium ion concentration.

The method V is in particular distinguished by the fact that the parameter for converting a bicarbonate ion concentration into a bicarbonate conductivity or vice versa, i.e. for converting a bicarbonate conductivity into a bicarbonate ion concentration, and the parameter for converting a sodium ion concentration into a conductivity of the dialysis fluid or vice versa, i.e. for converting a conductivity of the dialysis fluid into a sodium ion concentration, are both ascertained automatically.

Regarding further features and advantages of the method V, full reference is made to the discussions in the general description.

1. A method for determining a sodium and bicarbonate ion concentration of a dialysis fluid, the method comprising the steps of:
a) providing a dilute base fluid by mixing of a base fluid having a bicarbonate ion concentration with water according to a defined mixing ratio and providing a dilute acid fluid by mixing of an acid fluid having a sodium ion concentration and an acid concentration with water according to a defined mixing ratio;
b) preparing a pre-dialysis fluid by addition of the dilute base fluid to water;
c) determining a target value for a bicarbonate conductivity of the dialysis fluid and/or pre-dialysis fluid based on a bicarbonate ion concentration for the dialysis fluid and/or pre-dialysis fluid and a parameter for converting a bicarbonate ion concentration into a bicarbonate conductivity or vice versa and determining a target value for a conductivity of the dialysis fluid based on a sodium ion concentration for the dialysis fluid and a parameter for converting a sodium ion concentration into a conductivity of the dialysis fluid or vice versa;
d) measuring a conductivity of the pre-dialysis fluid;
e) comparing the conductivity of the pre-dialysis fluid measured according to step d) with the target value for the bicarbonate conductivity of the dialysis fluid and/or pre-dialysis fluid determined according to step c) and, if necessary, carrying out further addition of the dilute base fluid to the pre-dialysis fluid until the target value for the bicarbonate conductivity of the dialysis fluid and/or pre-dialysis fluid has been reached or substantially reached;
f) preparing the dialysis fluid by addition of the dilute acid fluid to the pre-dialysis fluid;
g) measuring a conductivity of the dialysis fluid;
h) comparing the conductivity of the dialysis fluid measured according to step g) with the target value for the conductivity of the dialysis fluid determined according to step c) and, if necessary, carrying out further addition of the dilute acid fluid to the dialysis fluid until the target value for the conductivity of the dialysis fluid has been reached or substantially reached; and
i) determining the bicarbonate ion concentration of the dialysis fluid based on the conductivity of the pre-dialysis fluid measured according to step d) and the parameter for converting a bicarbonate ion concentration into a bicarbonate conductivity or vice versa and determining the sodium ion concentration of the dialysis fluid based on the conductivity of the dialysis fluid measured according to step g) and the parameter for converting a sodium ion concentration into a conductivity of the dialysis fluid or vice versa,
wherein the parameter for converting a bicarbonate ion concentration into a bicarbonate conductivity or vice versa and the parameter for converting a sodium ion concentration into a conductivity of the dialysis fluid or vice versa are both ascertained automatically.

2. The method according to claim 1, wherein the base fluid is provided in the form of an aqueous basic solution comprising sodium hydrogencarbonate.

3. The method according to claim 1, wherein the acid fluid is provided in the form of an aqueous acidic solution comprising sodium chloride, potassium chloride, magnesium chloride, calcium chloride and an acid.

4. The method according to claim 1, wherein the target value for the bicarbonate conductivity of the dialysis fluid and/or pre-dialysis fluid is determined with a linear model according to the following equation (1):
$$BICLF = {c}_{bic} ( mmol / l ) \times {Λ}_{m , bic} \qquad ( 1 )$$
where
BICLF is the target value for the bicarbonate conductivity of the dialysis fluid and/or pre-dialysis fluid,
c_{bic} is the bicarbonate ion concentration for the dialysis fluid and/or pre-dialysis fluid and
Λ_{m,bic} is the parameter for converting a bicarbonate ion concentration into a bicarbonate conductivity or vice versa.

5. The method according to claim 1, wherein the target value for the conductivity of the dialysis fluid is determined with a linear model according to the following equation (2):
$$ENDLF = \{ [ {c}_{Na + , total} ( \text{mmol/l} ) - {c}_{bic} ( \text{mmol/l} ) ] \times {Λ}_{m , acid} \} + [ cbic ( \text{mmol/l} ) \times {Λ}_{m , bic} ] \qquad ( 2 )$$
where
ENDLF is the target value for the conductivity of the dialysis fluid,
c_{Na+,totat} is the sodium ion concentration for the dialysis fluid,
c_{bic} is the bicarbonate ion concentration for the dialysis fluid and/or pre-dialysis fluid,
Λ_{m,acid} is the parameter for converting a sodium ion concentration into a conductivity of the dialysis fluid or vice versa and
Λ_{m,bic} is the parameter for converting a bicarbonate ion concentration into a bicarbonate conductivity or vice versa.

6. The method according to claim 1, wherein the parameter for converting a bicarbonate ion concentration into a bicarbonate conductivity or vice versa and the parameter for converting a sodium ion concentration into a conductivity of the dialysis fluid or vice versa are ascertained automatically based on concentrations of the dilute base fluid and/or the dilute acid fluid.

7. The method according to claim 1, wherein the parameter for converting a bicarbonate ion concentration into a bicarbonate conductivity or vice versa is ascertained automatically based on a bicarbonate ion concentration of the dilute base fluid and an acid concentration of the dilute acid fluid.

8. The method according to claim 1, wherein the parameter for converting a sodium ion concentration into a conductivity of the dialysis fluid or vice versa is ascertained automatically based on a bicarbonate ion concentration of the dilute base fluid, a sodium ion concentration of the dilute base fluid, a sodium ion concentration of the dilute acid fluid, a potassium ion concentration of the dilute acid fluid, a magnesium ion concentration of the dilute acid fluid, a calcium ion concentration of the dilute acid fluid and an acid concentration of the dilute acid fluid.

9. The method according to claim 1, wherein the parameter for converting a bicarbonate ion concentration into a bicarbonate conductivity or vice versa is determined according to the following equation (3):
$${Λ}_{m , bic} = 0.0852 \frac{mS l}{mmol cm} \frac{{c}_{bic , 0} + {c}_{acid , 0} [ \frac{mEq}{l} ]}{{c}_{bic , 0}} \qquad ( 3 )$$
where
Λ_{m,bic} is the parameter for converting a bicarbonate ion concentration into a bicarbonate conductivity or vice versa,
c_{bic,0} is a bicarbonate ion concentration of the dilute base fluid and
c_{acid,0} is an acid concentration of the dilute acid fluid.

10. The method according to claim 1, wherein the parameter for converting a sodium ion concentration into a conductivity of the dialysis fluid or vice versa is determined according to the following equation (4):
$${Λ}_{m , acid , kor} = \frac{11 \frac{mS}{cm}}{{c}_{Na , {acid}^{+}} ( 11 \frac{mS}{cm} ) + {c}_{acid} ( 11 \frac{mS}{cm} )} \qquad ( 4 )$$
where Λ_{m,acid,kor} is the parameter for converting a sodium ion concentration into a conductivity of the dialysis fluid or vice versa,
$${c}_{Na , {acid}^{+}} ( 11 \frac{mS}{cm} )$$
is a sodium ion concentration of the dilute acid fluid that brings about a conductivity of the dilute acid fluid of 11 mS/cm,
and
$${c}_{acid} ( 11 \frac{mS}{cm} )$$
is an acid concentration of the dilute acid fluid having a sodium ion concentration which brings about a conductivity of the dilute acid fluid of 11 mS/cm.

11. The method according to claim 10, wherein the sodium ion concentration of the dilute acid fluid that brings about a conductivity of the dilute acid fluid of 11 mS/cm is determined according to the following equation (5):
$${c}_{Na , {acid}^{+}} ( 11 \frac{mS}{cm} ) = \frac{11 \frac{mS}{cm}}{{Λ}_{m , acid}} \qquad ( 5 )$$
where
$${c}_{Na , {acid}^{+}} ( 11 \frac{mS}{cm} )$$
is the sodium ion concentration of the dilute acid fluid that brings about a conductivity of the dilute acid fluid of 11 mS/cm, and
Λ_{m,acid} is an uncorrected parameter for converting a sodium ion concentration into a conductivity of the dialysis fluid or vice versa.

12. The method according to claim 11, wherein the uncorrected parameter Λ_{m,acid} for converting a sodium ion concentration into a conductivity of the dialysis fluid or vice versa is determined according to the following equation (6):
$${Λ}_{m , acid} = 0.09921 \frac{mS l}{mmol cm} \frac{{c}_{{Na}^{+} , 0} + {c}_{{K}^{+} , 0} + 2 * {c}_{{Mg}^{+} , 0} + 2 * {c}_{{Ca}^{+} , 0}}{{c}_{{Na}^{+} , 0}} \qquad ( 6 )$$
where
Λ_{m,acid} is the uncorrected parameter for converting a sodium ion concentration into a conductivity of the dialysis fluid or vice versa,
c_{Na}_{⁺}_{,0} is a sodium ion concentration of the dilute acid fluid,
c_{K}_{⁺}_{,0} is a potassium ion concentration of the dilute acid fluid,
2*c_{Mg}_{⁺}_{,0} is a magnesium ion concentration of the dilute acid fluid and
2*c_{Ca}_{⁺}_{,0} is a calcium ion concentration of the dilute acid fluid.

13. The method according to claim 10, wherein the acid concentration of the dilute acid fluid having a sodium ion concentration which brings about a conductivity of the dilute acid fluid of 11 mS/cm is determined according to the following equation (7):
$${c}_{acid} ( 11 \frac{mS}{cm} ) = \frac{{c}_{acid , 0} [ \frac{mEq}{l} ] * {c}_{Na , {acid}^{+}} ( 11 \frac{mS}{cm} )}{{c}_{{Na}^{+} , 0} - {c}_{bic , 0} - {c}_{acid , 0} [ \frac{mEq}{l} ]} \qquad ( 7 )$$
where
$${c}_{acid} ( 11 \frac{mS}{cm} )$$
is the acid concentration of the dilute acid fluid having a sodium ion concentration which brings about a conductivity of the dilute acid fluid of 11 mS/cm,
c_{bic,0} is a bicarbonate ion concentration of the dilute base fluid,
c_{acid,0} is an acid concentration of the dilute acid fluid,
c_{Na}_{⁺}_{,0} is a sodium ion concentration of the dilute acid fluid
and
$${c}_{Na , {acid}^{+}} ( 11 \frac{mS}{cm} )$$
is a sodium ion concentration of the dilute acid fluid that brings about a conductivity of the dilute acid fluid of 11 mS/cm.

14. The method according to claim 1, wherein concentrations of the base fluid and the acid fluid are stored on an extracorporeal blood treatment device.

15. The method according to claim 1, wherein at least part of the method is performed by an extracorporeal blood treatment device.

16. The method according to claim 1, wherein, depending on the bicarbonate ion concentration determined according to step i), a further addition of the dilute base fluid to the pre-dialysis fluid is carried out and/or, depending on the sodium ion concentration determined according to step i), a further addition of the dilute acid fluid to the dialysis fluid is carried out.

17. The method according to claim 1, wherein the dialysis fluid is prepared or proportioned using the method or haemodialysis is carried out using the method.
